(19) United States
(12) Patent Application Publication
(10) Pub. No.: US 2008/0138896 A1
(43) Pub. Date: Jun. 12, 2008

(54) Methods for Inhibiting Melanocyte Stem Cell Loss

(75) Inventors: Emi K. Nishimura, Ishikawa (JP); David E. Fisher, Newton, MA (US)

Correspondence Address: WOLF GREENFIELD & SACKS, P.C., 600 ATLANTIC AVENUE, BOSTON, MA, 02210-2206, US

(73) Assignee: Dana-Farber cancer Institute, Boston, MA (US)

(21) Appl. No.: 11/794,074

(22) PCT Filed: Dec. 22, 2005
(86) PCT No.: PCT/US05/46828

§ 371 Date: Oct. 25, 2007

Related U.S. Application Data
(60) Provisional application No. 60/639,062, filed on Dec. 23, 2004.

(51) Int. Cl. A61K 38/00; C12N 5/00; C12Q 1/02; A61K 31/70

(52) U.S. Cl. 435/375; 514/12; 435/29; 514/44; 435/6

(57) ABSTRACT

The invention relates to methods and compositions useful for altering and assessing melanocyte stem cell self-maintenance. Loss of such self-maintenance results in loss of melanocyte stem cells and loss of natural pigmentation of hair (e.g., graying hair). Methods for identifying candidate agents to inhibit melanocyte stem cell loss and to treat subjects with loss of natural hair pigment are also provided.

### FIELD OF THE INVENTION

The present invention relates generally to the field of stem cell biology, more particularly to melanocyte stem cell biology, and compositions and methods useful for inhibiting loss of follicular melanocyte stem cells involved in providing hair with its natural coloring.

### BACKGROUND OF THE INVENTION

Hair graying is an obvious sign of aging in man, yet its mechanism until now has been largely unknown. Qualitative and quantitative changes in stem/progenitor cells have been implicated in physiological (chronological) aging (M. A. Sussman, P. Anversa, Annu Rev Physiol 66:29-48 (2004); G. Van Zant, Y. Liang, Exp Hematol 31:659-72 (2003)), though the changes are poorly understood and the process of stem-cell aging has not been visually observed. While involvement of stem/progenitor cells in aging of multiple organ systems has been suggested in mice defective in DNA damage repair and telomere maintenance (K. K. Wong et al., Nature 421:643-8 (2003)), melanocytes may be unique in that the oxidative chemistry of melanin biosynthesis can be cytotoxic. K. Urabe et al., Biochim Biophys Acta 1221:272-8 (1994). It was recently reported that unpigmented melanocyte stem cells are distinctly located within the hair follicle. E. K. Nishimura et al., Nature 416:854-60 (2002).

Hair follicles contain a well-demarcated structure for the stem cell niche (within the lower permanent portion), whereas differentiated melanocytes reside in the hair bulb (at the base of the transient portion of the hair follicle) (FIG. 1a). E. K. Nishimura et al., Nature 416:854-60 (2002); E. Fuchs, B. J. Merrill, C. Jamora, R. DasGupta, Dev Cell 1, 13-25 (2001). Hair follicles are constantly renewing, with alternating phases of growth (anagen), regression (catagen), and rest (telogen) (FIG. 5).

It was previously reported that mice deficient in pro-survival, anti-apoptotic Bcl-2 turn gray with the second hair follicle cycle. D. J. Veis, C. M. Sorenson, J. R. Shutter, S. J. Korsmeyer, Cell 75:229-40 (1993). Hair-graying in the Bcl2^{-/-} background has been suggested to arise by chemical cytotoxity of melanin synthesis. J. P. Ortonne, J. J. Nordlund, in The Pigmentary System J. J. Nordlund, R. Boissy, V. J. Hearing, R. King, J. P. Ortonne, Eds. (Oxford University Press, New York, 1998) pp. 489-502; D. J. Veis, C. M. Sorenson, J. R. Shutter, S. J. Korsmeyer, Cell 75:229-40 (1993); K. Yamamura et al., Cancer Res 56, 3546-50 (1996); D. J. Tobin, R. Paus, Exp Gerontol 36:29-54 (2001).

### SUMMARY OF THE INVENTION

The present invention is based in part upon the discovery by the inventors that hair graying is caused by defective self-maintenance of melanocyte stem cells. This process is accelerated dramatically with Bcl-2 deficiency, which causes selective apoptosis of melanocyte stem cells within the niche at their entry into the dormant state. In contrast, Bcl-2 deficiency does not cause selective apoptosis of differentiated melanocytes. Loss of all melanocyte stem cells in a hair follicle leads to failure to repopulate differentiated melanocytes, so that as aging melanocytes are lost, the hair progressively loses its original natural color. Loss of some melanocytes results in graying hair, and, with essentially complete loss of melanocytes, white hair.

The present invention is also based in part on the discovery by the inventors that physiologic aging of melanocyte stem cells is associated with ectopic pigmentation or differentiation within the niche. This process can be accelerated and mimicked by mutation of the melanocyte master transcriptional regulator Mitf.

The invention provides methods and compositions useful for inhibiting loss of melanocyte stem cells, inhibiting ectopic pigmentation or differentiation within the niche, and screening for compounds that may be used to inhibit loss of melanocyte stem cells and to inhibit ectopic pigmentation or differentiation within the niche. Certain of the methods are useful for maintaining the natural color of hair. Certain of the methods are useful for treating loss of natural hair pigment.

The invention in one aspect is a method for inhibiting melanocyte stem cell loss. The method according to this aspect of the invention includes the step of contacting the melanocyte stem cells with an agent that inhibits melanocyte stem cell loss.

In one aspect the invention is a method for treating a subject having loss of natural hair pigment. The method according to this aspect of the invention includes the step of administering to the subject an agent that inhibits melanocyte stem cell loss in the subject.

The invention in one aspect is a method for identifying an agent that inhibits melanocyte stem cell loss. The method according to this aspect of the invention includes the steps of placing into culture a defined number of isolated epidermal melanocytes or isolated melanocyte stem cells, under conditions that simulate stem cell environment, for a defined duration in presence of a test agent; measuring a test response, wherein the test response is a number of epidermal melanocytes or melanocyte stem cells cultured for the defined duration in the presence of the test agent; comparing the test response to a control response, wherein the control response is a number of isolated epidermal melanocytes or isolated melanocyte stem cells, cultured beginning with the defined number of said cells, under the conditions that simulate stem cell environment for the defined duration in absence of the test agent; and identifying the test agent as an agent that inhibits melanocyte stem cell loss when the test response exceeds the control response.

In one aspect the invention is a method for identifying an agent that inhibits ectopic pigmentation or differentiation of melanocyte stem cells in a subject. The method according to this aspect of the invention includes the steps of administering a test agent to the subject; measuring a test response to the test agent, wherein the measuring comprises assessing ectopic pigmentation or differentiation of melanocyte stem cells in the subject; comparing the test response to a control response; and identifying the test agent as an agent that inhibits ectopic pigmentation or differentiation of melanocyte stem cells in the subject when the control response exceeds the test response.

The invention in one aspect is a method for identifying an agent that is useful for treating a subject having loss of natural hair pigment. The method according to this aspect of the invention includes the steps of placing into culture a defined number of isolated epidermal melanocytes or isolated melanocyte stem cells, under conditions that simulate stem cell environment, for a defined duration in presence of a test agent; measuring a test response, wherein the test response is a number of epidermal melanocytes or melanocyte stem cells cultured for the defined duration in the presence of the test agent; comparing the test response to a control response, wherein the control response is a number of isolated epidermal melanocytes or isolated melanocyte stem cells, cultured beginning with the defined number of said cells, under the conditions that simulate stem cell environment for the defined duration in absence of the test agent; and identifying the test agent as an agent that is useful for treating a subject having loss of natural hair pigment when the test response exceeds the control response.

In one aspect the invention is a method for identifying an agent that inhibits melanocyte stem cell loss in a subject. The method according to this aspect of the invention includes the steps of administering a test agent to the subject; measuring a test response to the test agent, wherein the measuring comprises counting melanocyte stem cells or assessing capacity of the melanocyte stem cells to produce melanocytes after entry into growth phase; comparing the test response to a control response; and identifying the test agent as an agent that inhibits melanocyte stem cell loss in the subject when the test response exceeds the control response.

In one embodiment the agent that inhibits melanocyte stem cell loss includes a cytokine. In one embodiment the agent that inhibits melanocyte stem cell loss is a cytokine.

In one embodiment the agent that inhibits melanocyte stem cell loss includes an agent that upregulates Mitf. In one embodiment the agent that inhibits melanocyte stem cell loss is an agent that upregulates Mitf.

In one embodiment the agent that inhibits melanocyte stem cell loss includes Mitf. In one embodiment the agent that inhibits melanocyte stem cell loss is Mitf.

In one embodiment the agent that inhibits melanocyte stem cell loss includes a nucleic acid encoding Mitf. In one embodiment the agent that inhibits melanocyte stem cell loss is a nucleic acid encoding Mitf.

In one embodiment the agent that inhibits melanocyte stem cell loss is not Bcl-2.

In one embodiment the agent that inhibits melanocyte stem cell loss includes an agent that upregulates Bcl-2. In one embodiment the agent that inhibits melanocyte stem cell loss is an agent that upregulates Bcl-2.

In one embodiment the agent that inhibits melanocyte stem cell loss includes a Bim antagonist. In one embodiment the agent that inhibits melanocyte stem cell loss is a Bim antagonist.

In one embodiment the agent that inhibits melanocyte stem cell loss is selectively targeted for delivery to melanocyte stem cells. In one embodiment the agent that inhibits melanocyte stem cell loss is conjugated to a Kit ligand.

In methods of the invention involving administration of a compound to a subject, in one embodiment the administering comprises topically administering. In one embodiment the administering is topically administering.

In methods of the invention involving administration of a compound to a subject, in one embodiment the administering comprises systemically administering. In one embodiment the administering is systemically administering.

In one embodiment the isolated epidermal melanocytes or isolated melanocyte stem cells are deficient for Bcl-2. In one embodiment the isolated epidermal melanocytes or isolated melanocyte stem cells are deficient for Mitf. Cells deficient for Mitf specifically include but are not limited to cells with a mutant form of Mitf, including Mitf^{vit/vit}.

In one embodiment the test agent includes a cytokine. In one embodiment the test agent is a cytokine.

In one embodiment the test agent includes an agent that upregulates Mitf. In one embodiment the test agent is an agent that upregulates Mitf.

In one embodiment the test agent includes Mitf. In one embodiment the test agent is Mitf.

In one embodiment the test agent includes a nucleic acid encoding Mitf. In one embodiment the test agent is a nucleic acid encoding Mitf.

In one embodiment the test agent is not Bcl-2.

In one embodiment the test agent includes an agent that upregulates Bcl-2. In one embodiment the test agent is an agent that upregulates Bcl-2.

In one embodiment the test agent includes a Bim antagonist. In one embodiment the test agent is a Bim antagonist.

In one embodiment the test agent is selectively targeted for delivery to melanocyte stem cells. In one embodiment the test agent is conjugated to a Kit ligand.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 is a panel of 9 figures showing that differentiated melanocytes are lost in the hair bulb of Bcl-2 deficient mice. a, Hair Follicle Structure. Melanocyte stem cells are in the lower permanent portion: the bulge area (Bg) in pelage follicles and the lower enlargement (LE) in whisker follicles. Additional features indicated in the figure include arrector pili muscle (APM); capsule (C); hair shaft (HS); hair matrix (M); peripheral nerve (N); outer root sheath (ORS); and sebaceous gland (S). b, Appearance of Bcl2^{-/-} mouse at postnatal day 58 (P58). c, Hair graying of whiskers in Bcl2^{-/-} mouse at P39. d, e, Distribution of lacZ⁺ cells (melanocytes) in P39 Bcl2^{-/+} and Bcl2^{-/-} mice, carrying the Dct-lacZ transgene. Pigmented melanocytes in the bulb (Bb) (d: arrows) and lacZ⁺ melanoblasts in the bulge (Bg) (d: arrowhead; the inset shows the magnified view) are completely lost in Bcl2^{-/-} follicles (e). Double arrows indicate the level of Bb or Bg. Magnification: ×100. f-i, Whole mount lacZ staining of the bulb of whisker follicles from Bcl2^{-/+} and Bcl2^{-/-} in black and white/albino (Tyr^{c-2j/c-2j}) backgrounds at P40. Loss of Dct-lacZ⁺ melanocytes was detected in the bulb of Bcl2^{-/-} whisker follicles regardless of albino background.

FIG. 2 is a series of 12 photomicrographs and a bar graph depicting loss of Bcl2^{-/-} melanocyte-stem cells upon entry into the dormant state. a-d, Distribution of Dct-lacZ⁺ melanoblasts (arrows) in the bulge (Bg) (upper double arrow) of pelage follicles at P6.5 and P8.5. While bulb (Bb) melanocytes appear largely unchanged (lower double arrow), bulge melanoblasts are lost in Bcl2^{-/-} follicles at P8.5 (compare d to c), but not at P6.5 (compare b to a). e, Comparison of the total number per field of Dct-lacZ⁺ melanoblasts in the bulb and in the bulge/sub-bulge of Bcl2^{-/-} and Bcl2^{-/+} pelage follicles at P8.5 on 7 μm sections (magnified 100×). f, g, KIT expression matches Dct-lacZ⁺ in bulge melanoblasts (stage 6 (f) and stage 8 (g)) of Bcl2^{+/+} animals (X630). Cell size is diminished from stage 6 to stage 8. h, i, TUNEL/LacZ/DAPI staining of stage 7 skin from P6.5 Bcl2^{-/-} (h) and P6.5 Bcl2^{+/-} (i). Arrowheads show apoptotic inner root sheath keratinocytes. The inset pointed with the arrow in (h) shows an apoptotic melanoblast. The upper inset on the right in h shows the merged view for TUNEL (bright) and DAPI (background). The lower inset on the right in h shows the merged view for TUNEL (background) and LacZ (bright). The arrow in i points to bright staining for LacZ. j-m, Distribution of Dct-lacZ⁺ melanoblasts in the niche: Bg of pelage hair follicles (j, k) and lower enlargement (LE) (double arrow) of whisker hair follicles (l, m; double arrows) from the mice of P8.0 with white background (Tyr^{c-2j/c-2j}).

FIG. 3 is a series of 7 photomicrographs and two graphs depicting the effect of aging and Mtif mutation on melanocyte stem cells. a, b. Coincident expression of Dct-LacZ, KIT, and MITF in hair follicle melanoblasts/melanocytes (X200). Arrowheads, KIT; arrows, LacZ; other bright staining, DAPI; Bg, bulge; Bb, bulb. c. Ectopically pigmented melanoblasts (lacZ⁺=arrows) in the bulge region (Bg) of 3.5 month old Mitf^{vit/vit} follicles. d. Magnified view of pigmented bulge melanoblasts. e. Absence of pigment in lacZ+ bulge melanoblasts of age-matched Mitf/^{+/+} follicles. f, Quantitation of niche melanocytes (LacZ⁺), either unpigmented (classical stem cells, lacZ+ pigment−) or ectopically pigmented (lacZ+ pigment+) in lower enlargement (LE) of whisker follicles (position a2, a3). g, Number of unpigmented niche melanoblasts in whisker follicles (position a2, a3, c5, d5) with black, gray and white hair in 18-22M wildtype (wt) mice. *indicates statistical significance (P<0.01). h, i. Ectopically pigmented melanoblasts in the niche (lower enlargement (LE) of whisker follicles) of aging wildtype mice (whole mount view).

FIG. 4 is a series of 8 photomicrographs, a drawing, and a bar graph depicting melanoblast/melanocyte distribution in human hair follicles from different age groups. Human scalp specimens were immunostained with anti-MITF antibody. a, b, MITF⁺ cells (arrows) are distributed on the outer root sheath in the bulge of follicles from 20-30 year olds. a: X200, b:X630. c, d, Representative views of the bulge from follicles of 40-60 and 70-90 year old people, respectively (X630). MITF⁺ cells are indicated with *indicates statistical significance (P<0.01). yo; years old. arrows. e, Schematic for human hair follicle with pigmented hair. Immature MITF^{low} melanoblasts are located in the lower permanent portion (Bg, the bulge). MITF^{high} melanocytes are located in the epidermis, infundibulum (If) and hair matrix (M). P: permanent portion, T: transient portion, Bb: hair bulb. f, g, h, The bulb region of follicles from different age groups. Mature melanocytes in the hair matrix express MITF (arrowheads). i, An MITF⁺ melanocyte (arrow) which contains abundant melanin granules and long dendrites is detected in the bulge/sub-bulge of follicles specifically from middle-aged individuals. j, The frequency of MITF⁺ cells per basal keratinocytes in the bulge. *indicates statistical significance (P<0.01). yo; years old.

FIG. 5 is a series of 6 drawings and 7 photomicrographs. a, Schematic for melanocyte lineage regeneration coupled with the pelage-hair cycle. SC: melanocyte stem cells, AC: amplifying cells, MC: mature melanocytes. P: permanent portion, T: transient portion, Bg: bulge area, Bb: hair bulb. b-h, Melanocyte regeneration cycle coupled with the whisker-hair cycle. Double-arrows indicate the position of lower enlargement (niche) in whisker follicles. The whisker follicles from position c5/d5 were examined at different stages from the second to third hair cycles. i, Location of whisker (vibrissal) follicles of the mystacial pad of the rat (modified from the scheme from M. A. Mackenzie, S. A. Jordan, P. S. Budd, I. J. Jackson, Dev Biol 192, 99-107 (1997)). Whisker follicles occupying the positions a2, a3, c5, d5 were used.

FIG. 6 is a series of 16 photomicrographs depicting melanoblast/melanocyte distribution in Bcl2^{-/-} and Bcl-2^{-/+} mice carrying the Dct-lacZ transgene. a-h, Melanoblast colonization of the skin is intact in Bcl-2^{-/-} and Bcl-2^{-/30} embryos. Whole mount staining of Bcl-2^{-/30} (a, c, e, and g) and Bcl-2^{-/-} (b, d, f, and h) embryos of E13.5. e and f show magnified view of the surface skin above the ear. g and h show a cross section of the developing whisker pad. Melanoblasts are migrating into the whisker hair buds (border marked by dashes). i, j, Magnified views of FIG. 1d and 1e, respectively. X200. k-n, Loss of Bcl2^{-/-} melanoblasts in the stem cell niche (LE: lower enlargement) in whisker hair follicles. Whole mount lacZ staining of the whisker hair follicles of Bcl2^{-/+} (k and m) and Bcl-2^{-/-} (l and n) at E16.5 (k and l) and P0.5 (m and n). Disappearance of Dct-lacZ⁺ melanoblasts started from E16.5 (l) and the loss was almost complete by birth (n) in Bcl2^{-/-} . o, p, Epidermal and dermal melanocytes are maintained intact in Bcl-2^{-/-} mice. Skin section of the tail shows that both epidermal melanocytes (arrow) and dermal melanocytes (arrowheads) are intact in Bcl-2^{-/-} mice even at P39.

FIG. 7 is a series of 10 photomicrographs showing concordant loss of KIT/MITF/Dct-lacZ expression in the niche of mouse anagen follicles in Bcl2 deficient mice. a, b, Immunostaining for MITF/LacZ of activated melanoblasts in the bulge (Bg) (a) and in the hair bulb (Bb) (b). MITF is localized in the nucleus of the Dct-lacZ positive cells. c-j, MITF and LacZ expression (c-f) and KIT and LacZ expression (g-j) in pelage follicles from Bcl1^{-/-} and Bcl2^{-/+} mice at P8. c, d, g, h, MITF⁺LacZ⁺KIT⁺ cells detected in the bulge area of follicles in Bcl2^{-/+} mice (arrows) were not found in Bcl2^{-/-} mice. e, f, i, j, MITF⁺LacZ⁺KIT⁺melanocytes (arrowheads) were found in the bulb of follicles from both Bcl2^{-/-} and Bcl2^{-/+} mice. Keratinocytes located at the tip of bulb region of mid-anagen follicles are KIT⁺ (arrows) as previously described (Peters et al., J Invest Dermatol 121, 976-984, 2003). Bg: bulge area, Bb; bulb.

FIG. 8 is a series of 21 photomicrographs and photographs depicting loss of melanocyte stem cells at different stages in Mitf^{vit/vit} and Mitf^{vit/+} compared to Mitf^{+/+} mice. a-o, Whole mount lacZ staining of whisker follicles (position a2/a3) at the indicated stages. The stem-cell niche in whisker follicles is highlighted by a double-arrow. In Mitf^{vit/vit} mice, LacZ⁺ melanoblasts which have colonized the presumptive stem-cell niche by E16.5 showed a gradual but progressive decrease in number resulting in their complete disappearance at P13 (k-m) and the growth of white whiskers during the following hair cycle (n). Mitf^{vit/+} animals showed an even more gradual decrease of niche melanoblasts than in Mitf^{vit/vit} mice after normal colonization of the niche, and showed a further gradual decrease with hair cycling (f-j). E: embryonic day, M: month. p, Appearance of Mitf^{vit/vit} at 1 and 9 months old. q, Whisker of 6 month old Mitf^{vit/+} mouse. Mitf^{vit/+} mutants often show mild whisker graying starting at 6 months in addition to an occasional congenital belly spot. r, Whole mount lacZ staining of the whisker follicles from 6 month old Mitf^{vit/+} (r). s, Magnified views (X630) of lacZ⁺ melanoblasts in the lower enlargement of whisker follicles from Mitf^{+/+} and Mitf^{vit/+} mice at P44. LacZ⁺ cells in the niche extend a long dendrite and contain melanin pigment in Mitf^{vit/+} (arrowheads) but not in Mitf^{+/+} follicles at P44. t, u, LacZ staining of the trunk skin section from 3.5 month old Mitf^{+/+} and Mitf^{vit/vit}. LacZ ⁺cells in the bulge (Bg) are pigmented in Mitf^{vit/vit} but not in Mitf^{+/30} mice. Bb: bulb.

FIG. 9 is a series of 14 photographs and photomicrographs depicting ectopic pigmentation or differentiation and gradual loss of melanocyte stem-cells in the niche with physiological aging. a, Hair graying in 18 month old (18M) and 6M wildtype (wt) mice (C57BL/6J) (arrowheads indicate white hairs). Whisker graying was found frequently during backcrossing from a mixed background of CBA/C57BL/6 to C57BL/6, while pelage hair graying occurred earlier in pure C57BL/6. b. White whisker in 18M wildtype. c-j, Whole mount lacZ staining (d, f, h, j) and non-staining view (c, e, g, i) of wildtype whisker follicle at mid-anagen of different ages. Double-arrows show the niche (the lower enlargement: LE) in whisker follicles. i, Non-staining view of C57BL/6J whisker follicles. k-n, Magnified view (X400) of lacZ⁺ cells (arrows) in the upper (k, l) and the lower area (m, n) of LE. Pigmented melanocytes in the lower area of the niche contain various quantities of melanin in the cytoplasm and oval-bipolar-dendritic morphology with variable levels of Dct-lacZ expression (n). N: sensory nerve endings. LacZ⁺ cells in the niche contain abundant melanin at 8M.

FIG. 10 is a series of 19 photomicrographs depicting concordant loss of KIT/MITF/Dct-lacZ expression in the niche during the process of hair graying. Immunostaining of pelage follicles (a-g) and whisker follicles (h-r) for MITF/KIT and LacZ. a-c shows immunostaining for MITF and LacZ. MITF⁺LacZ⁺ cells are found in the bulge (arrows) and bulb (hair matrix) (arrowheads) of anagen follicles both in wildtype mice and Mitf^{vit/vit} mutant mice at 3.5 months old. Background (asterisk) with secondary antibody only was preferentially seen on the basement membrane (c). d-g KIT⁺LacZ⁺ cells are found in the bulge and the bulb of wildtype follicles (d) and Mitf^{vit/vit} mutant follicles (f, g), while some Mitf^{vit/vit} mutant follicles have lost KIT⁺LacZ⁺ cells in the bulge area (e). There is some variation in precise expression intensity for each of the three markers in individual melanoblasts (d), suggesting that expression of these molecules is independent of each other to some extent. h-s, MITF/LacZ or KIT/LacZ staining of whisker follicles. The lower enlargement is shown. Background (asterisk) with secondary antibody only was preferentially seen on the basement membrane (I). MITF and KIT expression were detected in Dct-lacZ expressing melanoblasts in the lower enlargement at different ages both in wildtype and Mitf^{vit/+} mutant follicles. MITF⁺LacZ⁺ melanoblasts (h, i) and KIT⁺LacZ⁺ melanoblasts (n, o) were found in the lower enlargement of whisker follicles at 3 and 6 months, while cells were absent or rare in 2 year old (j, p) and 1 year old Mitf^{vit/+} mutant follicles (k, q, r). s, KIT⁺LacZ⁺ melanocytes which assume dendritic morphology were occasionally found in the lower enlargement of 2 year old follicles. Bg: bulge area, Bb; bulb.

FIG. 11 is a bar graph depicting the frequency of MITF⁺ cells per basal keratinocytes in the bulge area and infundibulum of human hair follicles from different age groups. Stippled and white bars show the frequency in the bulge area and infundibulum, respectively. yo; years old.

### DETAILED DESCRIPTION OF THE INVENTION

Stem cells, which have the capacity to self-renew and generate differentiated progeny, are thought to be maintained in a specific environment known as a niche. The localization of the niche, however, remains largely obscure for most stem-cell systems. Melanocytes (pigment cells) in hair follicles proliferate and differentiate closely coupled to the hair regeneration cycle. Stem cells of the melanocyte lineage previously were identified, using Dct-lacZ transgenic mice, in the lower permanent portion of mouse hair follicles throughout the hair cycle. E. K. Nishimura et al., Nature 416:854-60 (2002). It is only the population in this region that fulfils the criteria for stem cells, being immature, slow cycling, self-maintaining and fully competent in regenerating progeny on activation at early anagen (the growing phase of hair follicles).

Hair regeneration initiates at early anagen from the bulge area, where stem cells of the follicular keratinocytes reside. This is followed by downward growth of the basal portion of the hair follicles, the hair matrix. Subsequently, these follicles regress at catagen and become resting at telogen. Melanocytes in the hair matrix proliferate in anagen, differentiate to produce melanin pigment that is transferred to hairs, and then die by apoptosis during catagen. The stem-cell system is divided into three compartments: stem cells, transiently amplifying cells, and mature cells. The latter two compartments of the melanocyte lineage reside in the hair matrix.

As used herein, melanocyte stem cells refer to unpigmented Dct⁺ stem cells which give rise to differentiated, pigmented melanocytes. These cells are normally found in hair follicles in a well-demarcated structure for the stem cell niche with the lower permanent portion of the follicle. Melanocyte stem cells are also referred to herein as melanoblasts.

As used herein, melanocyte stem cell loss refers to a decrease in the absolute number of melanocyte stem cells in a site or under conditions where melanocyte stem cells normally survive. In one embodiment melanocyte stem cell loss refers to a complete loss of melanocyte stem cells in a site or under conditions where melanocyte stem cells normally survive. Melanocyte stem cell loss is usually attributable to death of the melanocyte stem cells. In one embodiment melanocyte stem cell loss is attributable to apoptotic death of melanocyte stem cells.

As described in greater detail below, it has now been discovered by the inventors that hair graying is a manifestation of incomplete self-maintenance of melanocyte stem cells. In one model (Bcl-2 deficiency) all or essentially all melanocytes simply die off, leaving no residual melanocyte stem cells to generate new melanocytes. Graying comes about suddenly in this model. In another model, which is more like natural aging, there occur gradually and simultaneously both a decrease in the population of normal melanocyte stem cells and the appearance of ectopically pigmented and differentiated melanocytes within the niche. These abnormal appearing cells within the niche represent cells that have lost their ability to replenish fully differentiated melanocytes, i.e., they no longer act as stem cells.

Measurement of melanocyte stem cells can be performed using any suitable method, including microscopically or by gross inspection. In one embodiment melanocyte stem cells are counted with the aid of a microscope and, optionally, a suitable dye or marker specific for melanocytes. For example, Dct is an enzyme involved in the synthesis of pigment in melanocytes. As another example, D5 is a monoclonal antibody specific for melanocytes. The melanocyte stem cells are characteristically unpigmented but are identifiable with Dct or with D5. Yet additional markers useful according to the invention include Kit and PMEL17.

As used herein, an agent that inhibits melanocyte stem cell loss refers to any of a variety of compositions capable of maintaining melanocyte stem cell viability. In one embodiment the agent is a cytokine. Cytokines as used herein include interleukins, chemokines, interferons, colony stimulating factors (CSFs), tumor necrosis factor (TNF), and stem cell factor (c-Kit ligand, SCF).

An agent that inhibits melanocyte stem cell loss may advantageously be used in combination with another agent that may protect against aging. In one embodiment such other agent is a vitamin, e.g., vitamin C or vitamin E. In one embodiment such other agent is an antioxidant agent other than a vitamin.

In one embodiment an agent that inhibits melanocyte stem cell loss refers to an agent that upregulates Mitf. Mitf is a basic helix-loop-helix leucine zipper (b-HLH-Zip) transcription factor encoded by the microphthalmia gene. Mitf has been reported to be a master regulator of transcription in melanocytes. Bcl-2 has been reported to be regulated by Mitf. McGill GG et al. Cell 109:707-718 (2002). In one embodiment the agent that upregulates Mitf is a nucleic acid molecule encoding Mitf. Introduction of the nucleic acid molecule encoding Mitf into a host cell can result in de novo expression or increased expression of Mitf by that cell. The nucleic acid molecule encoding Mitf is in one embodiment operably incorporated into an expression vector.

In one embodiment Mitf is a human Mitf. In one embodiment a human Mitf polypeptide has a sequence provided as GenBank Accession No. NP_{—}000239, the entire content of which is incorporated herein by reference. In one embodiment a nucleic acid molecule encoding a human Mitf has a sequence that encodes a human Mitf polypeptide has a sequence provided as GenBank Accession No. NP_{—}000239. In one embodiment a nucleic acid molecule encoding a human Mitf has a sequence provided as nucleotides 122-1378 of GenBank Accession No. NM_{—}000248, the entire content of which is incorporated herein by reference.

In one embodiment Mitf is a murine Mitf. In one embodiment a murine Mitf polypeptide has a sequence provided as GenBank Accession No. NP_{—}032627, the entire content of which is incorporated herein by reference. In one embodiment a nucleic acid molecule encoding a murine Mitf has a sequence that encodes a murine Mitf polypeptide has a sequence provided as GenBank Accession No. NP_{—}032627. In one embodiment a nucleic acid molecule encoding a murine Mitf has a sequence provided as nucleotides 130-1386 of GenBank Accession No. NM_{—}008601, the entire content of which is incorporated herein by reference.

The nucleic acid encoding Mitf is operably linked to a gene expression sequence which directs the expression of the Mitf nucleic acid within a eukaryotic cell. The gene expression sequence is any regulatory nucleotide sequence, such as a promoter sequence or promoter-enhancer combination, which facilitates the efficient transcription and translation of the Mitf nucleic acid to which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter, and other constitutive promoters. Exemplary viral promoters which function constitutively in eukaryotic cells include, for example, promoters from the cytomegalovirus (CMV), simian virus (e.g., SV40), papillomavirus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined Mitf nucleic acid. The gene expression sequences optionally include enhancer sequences or upstream activator sequences as desired.

The Mitf nucleic acid is operably linked to the gene expression sequence. As used herein, the Mitf nucleic acid sequence and the gene expression sequence are said to be operably linked when they are covalently linked in such a way as to place the expression or transcription and/or translation of the Mitf coding sequence under the influence or control of the gene expression sequence. Two DNA sequences are said to be operably linked if induction of a promoter in the 5′ gene expression sequence results in the transcription of the Mitf sequence and if the nature of the linkage between the two DNA sequences does not (1) result in the introduction of a frame-shift mutation, (2) interfere with the ability of the promoter region to direct the transcription of the antigen sequence, or (3) interfere with the ability of the corresponding RNA transcript to be translated into a protein. Thus, a gene expression sequence would be operably linked to a Mitf nucleic acid sequence if the gene expression sequence were capable of effecting transcription of that Mitf nucleic acid sequence such that the resulting transcript is translated into the desired protein or polypeptide.

The Mitf nucleic acid of the invention may be delivered to a cell alone or in association with a vector. In its broadest sense, a vector is any vehicle capable of facilitating the transfer of the Mitf nucleic acid to the cells so that the Mitf can be expressed by the cell. The vector generally transports the nucleic acid to the cells with reduced degradation relative to the extent of degradation that would result in the absence of the vector. The vector optionally includes the above-described gene expression sequence to enhance expression of the Mitf nucleic acid in cells. In general, the vectors useful in the invention include, but are not limited to, plasmids, phagemids, viruses, other vehicles derived from viral or bacterial sources that have been manipulated by the insertion or incorporation of the antigen nucleic acid sequences. Viral vectors are a preferred type of vector and include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyoma viruses; Epstein-Barr viruses; papillomaviruses; herpes virus; vaccinia virus; polio virus; and RNA virus such as a retrovirus. One can readily employ other vectors not named but known in the art.

Certain viral vectors are based on non-cytopathic eukcaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murray, E. J., Methods in Molecular Biology, vol. 7, Humana Press, Inc., Cliffton, N.J. (1991).

A virus for certain applications is the adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hematopoietic cells; and lack of superinfection inhibition thus allowing multiple series of transductions. Reportedly, wild-type adeno-associated virus manifest some preference for integration sites into human cellular DNA, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion. Recombinant adeno-associated viruses that lack the replicase protein apparently lack this integration sequence specificity.

Other vectors include plasmid vectors. Plasmid vectors have been extensively described in the art and are well-known to those of skill in the art. See e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRc/CMV, SV40, and pBlueScript. Other plasmids are well-known to those of ordinary skill in the art. Additionally, plasmids may be custom designed using restriction enzymes and ligation reactions to remove and add specific fragments of DNA.

In one embodiment an agent that inhibits melanocyte stem cell loss refers to Mitf polypeptide. In one embodiment the Mitf is an isolated recombinantly expressed Mitf.

In one embodiment an agent that inhibits melanocyte stem cell loss refers to an agent that upregulates Bcl-2. The agent that upregulates Bcl-2 is in one embodiment a nucleic acid encoding Bcl-2. The nucleic acid molecule encoding Bcl-2 is in one embodiment operably incorporated into an expression vector.

In one embodiment an agent that inhibits melanocyte stem cell loss refers to a Bim antagonist. Bim is a pro-apoptotic member of the Bcl-2 family. Bim includes various pro-apoptotic isoforms of Bim (e.g., BimS, BimL, and BimEL). Additional pro-apoptotic members of the Bcl-2 family include Bax, Bak, Bok, Bad, Bid, Bik, Blk, Hrk, and BNIP3.

As used herein, a subject having loss of natural hair pigment refers to a subject in which natural hair color is changing or has changed to gray or white. Hair color is normally determined at least in part by the amount of pigment (melanin) that is incorporated into the growing hair. With progressive loss of pigmentation, hair color turns gray (reflective of partial pigmentation) and eventually to white (reflective of essentially complete loss of pigmentation. In one embodiment a subject having loss of natural hair pigment is a subject with gray or graying hair. In one embodiment a subject having loss of natural hair pigment is a subject with white hair. In one embodiment a subject having loss of natural hair pigment is a subject with gray or graying hair and white hair. Natural pigment is to be distinguished from applied or cosmetic hair coloring.

In certain embodiments the agent that inhibits melanocyte stem cell loss is selectively targeted for delivery to melanocyte stem cells. Any suitable target found on or in a melanocyte stem cell can be used for this purpose. It is reported, for example, that melanocyte stem cells express Kit receptor (also known as c-Kit receptor). Accordingly in one embodiment the agent that inhibits melanocyte stem cell loss is conjugated to a Kit ligand. The resulting conjugate is believed to be taken up by the Kit receptor, thereby selectively delivering the agent to the melanocyte stem cell. In another embodiment, particularly where the agent is a nucleic acid encoding a protein or polypeptide, the agent is conjugated to a nucleic acid sequence specific for melanocytes, e.g., a promoter sequence for Dct. The conjugate can be made using any suitable physicochemical method that results in a conjugate capable of selective delivery of agent without seriously compromising an inhibitor function of the agent.

Certain aspects of the invention involve administration of an agent to a subject. The route of administration can be any suitable route of administration that can be used to effect contact between the agent and melanocyte stem cells in the subject. In one embodiment the route of administration is systemic. Systemic delivery includes oral and parenteral routes, the latter specifically including but not limited to intravenous, intramuscular, intradermal, transdermal, subcutaneous, intraperitoneal, and mucosal (e.g., intranasal, intrapulmonary, intravaginal, rectal). In one embodiment the route of administration is topical, including in particular topical administration to skin.

In certain aspects of the invention methods are provided for screening for agents that inhibit melanocyte stem cell loss. Such methods can in certain embodiments be adapted to be performed as high-throughput methods. As used herein high throughput refers to the serial or parallel performance of multiple assays such that the rate of assay performance significantly exceeds a corresponding rate performed by a standard or non-high throughput manner. For example, the rate of assay performance can be at least two-fold greater, and more typically 10- to 1000-fold greater than the rate performed by a standard or non-high throughput manner. High throughput screening frequently achieves assay rates of tens, hundreds, thousands, or even tens of thousands of assays performed in a single day. General methods and devices useful for performing high throughput screening are well known in the art. These include the use of multiwell plates, robotic sample handling devices, multichannel analyzers of various types, and the like.

Test agents useful in screening methods of the invention generally can include, but are not limited to, cells, cell extracts, proteins, polypeptides, peptides, antibodies, antigen-specific antibody fragments, polysaccharides, polysaccharide conjugates, peptide and non-peptide mimics of polysaccharides and other molecules, nucleic acids, small molecules (i.e., molecular weight less than about 1.5 kDa), lipids, glycolipids, and carbohydrates.

Certain methods of the invention use isolated epidermal melanocytes or isolated melanocyte stem cells. As used herein a composition is said to be isolated when it has been being removed from an environment in which it occurs or is found in nature.

Further with respect to use of isolated epidermal melanocytes or isolated melanocyte stem cells, it is believed that epidermal melanocytes represent a rather unique phenotype that represents a sort of hybrid between a pure stem cell and a pure differentiated melanocyte. Such cells can be isolated from foreskin. Culture conditions can be selected to emulate a stem cell environment, so that the epidermal melanocytes can be used as a sort of surrogate melanocyte stem cell. Because melanocyte stem cells are quite rare (e.g., only a few per pelage follicle, while epidermal melanocytes are relatively far more numerous, the use of isolated epidermal melanocytes may afford certain practical advantages over the use of isolated melanocyte stem cells.

As used herein, the terms treat and treating refer to preventing, reducing, or eliminating at least one symptom or sign of a disease or condition in a subject. Thus for example treating a subject having loss of natural hair pigment refers to preventing, reducing, or eliminating at least one symptom or sign of loss of natural hair pigment in a subject.

As used herein, a subject refers to a vertebrate animal. In one embodiment the subject is a human. In certain other non-limiting embodiments a subject is a mouse, a rat, a rabbit, a guinea pig, a dog, a cat, a sheep, a goat, a horse, or a cow.

The methods of the invention involve the use of effective amounts of various inhibitors. The term effective amount refers generally to the amount necessary or sufficient to realize a desired biologic effect. A therapeutically effective amount, as used herein, refers to the amount necessary or sufficient to realize a desired therapeutic effect, i.e., to treat a subject having a condition or disease. The therapeutically effective amount can vary depending on the route of administration, the formulation, the disease or condition being treated, the particular active agent being administered, the size of the subject, or the severity of the disease or condition. A therapeutically effective amount can be administered as one or more doses.

Combined with the teachings provided herein, by choosing among the various active compounds and weighing factors such as potency, relative bioavailability, patient body weight, severity of adverse side-effects, and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular inhibitor being administered, the size of the subject, or the severity of the disease or condition. One of ordinary skill in the art can empirically determine the effective amount of a particular inhibitor and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

For any compound described herein the therapeutically effective amount can be initially determined from animal models. A therapeutically effective dose can also be determined from human data for inhibitors which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The inhibitors and optionally additional agents may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of an inhibitor in a pharmaceutically-acceptable carrier. The term “pharmaceutically-acceptable carrier” means one or more compatible solid or liquid fillers, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

Compositions suitable for parenteral administration conveniently comprise sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose any bland fixed mineral or non-mineral oil may be employed including synthetic mono- or di-glycerides. In addition, fatty acids such as oleic acid find use in the preparation of injectables. Carrier formulations suitable for subcutaneous, intramuscular, intraperitoneal, intravenous, etc. administrations may be found in Remington's Pharmaceutical Sciences, Mack Publishing Company, Easton, Pa.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The following examples provide a new pathophysiologic explanation for hair graying. Loss of melanocyte stem cells can be observed, and temporally precedes the loss of differentiated melanocytes in the hair matrix. Thus incomplete maintenance of melanocyte stem cells appears to cause physiologic hair graying through loss of the differentiated progeny with aging. This is associated with “ectopic melanocyte pigmentation or differentiation” within the niche. Possible explanations include premature differentiation or activation of a senescence program (which induces pigmentation in vitro (E. E. Medrano et al., Mol Biol Cell 5:497-509 (1994))). Acceleration of this process in Mitf^{vit} follicles implicates MITF in the self-renewal of melanocyte stem-cells.

### Example 1

### General Methods

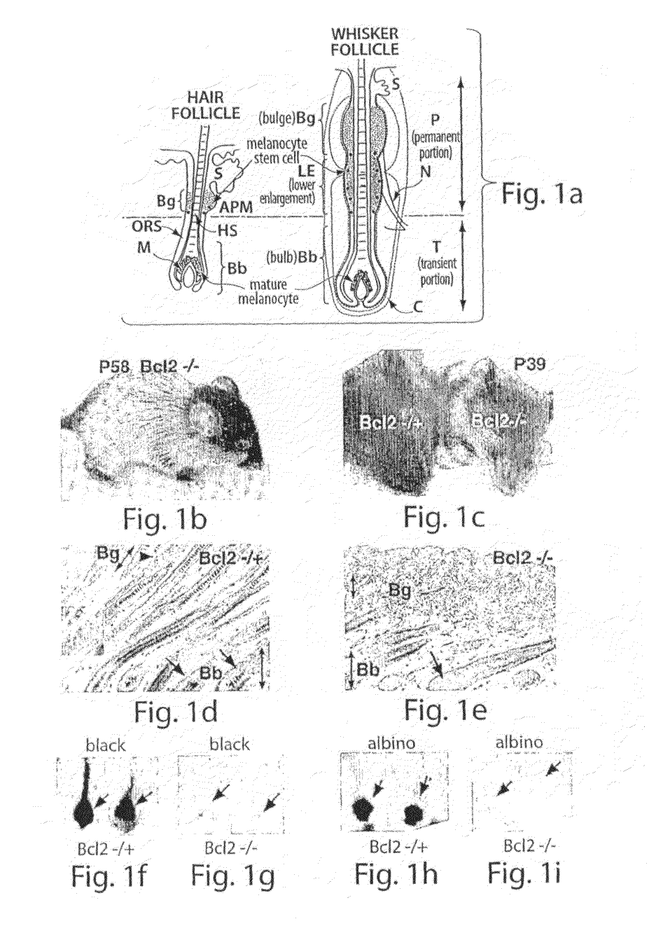

Whole mount β-galactosidase staining. Skin samples from Dct-lacZ transgenic mice were immersed in fixation solution (2% formaldehyde, 0.2% glutaraldehyde, 0.02% nonidet P-40 in phosphate-buffered saline (PBS) (pH 7.4)), irradiated for 20 s in a 600 W microwave oven at 4° C. and kept on ice for 40-60 min, while the thick adipose tissue in adult mice was removed and the whisker follicles were dissected out with micro-dissecting scissors under a dissecting microscope. The fixed samples were stained in 5-bromo-4-chloro-3-indolyl-B-D-galactoside (X-gal) (Invitrogen, Calif.) solution. The stained tissues were postfixed with 10% formalin solution and the image was captured with a SZH10 microscope (Olympus)-mounted SPOT digital camera (Diagnostic Instruments Inc.). For quantitation of niche melanocytes, the LacZ⁺ cell number was counted on the side opposite the major sensory nerve endings (opposite “N” depicted in FIG. 1a).

Immunohistochemistry for mouse skin. Skin samples were immersed in 2% paraformaldehyde/PBS (pH7.4), irradiated in a 600 W microwave oven for 20-60 seconds at 4° C. and kept at 4° C. for 10-20 min. The fixed skin was embedded in OCT compound and snap frozen. For double immunofluorescent staining, 8 μm cryosections were processed in the following solutions: (a) PBSMT (2% skim milk powder and 0.1% v/v Triton X-100 in PBS) for 20 min at room temperature; (b) rat anti-Kit (ACK2) (1:150) or mouse anti-MITF (C5), and rabbit anti-β-galactosidase (Cappel, Aurora, Ohio) (1:900) antibodies in PBST (0.1% v/v Triton X-100 in PBS) overnight; (c) Alex594-conjugated anti-rabbit (Molecular Probes, Inc., Eugene, OR) (1:900) and FITC-conjugated anti-rat or mouse (Molecular Probes) (1:450) in PBST for 2 hr. The specimens were washed three times with PBST between steps and then mounted with slow-fade reagent (Molecular Probes). Images were captured with the Axioplan2 imaging system or Axiovert 200M (Carl Zeiss). Images were deconvoluted using slidebook software (Intelligent Imaging Innovations, Inc., Denver, Colo.)

Immunohistochemistry for human skin. Human scalp specimens which include intact/normal human scalp follicles were selected from the pathology files of the department of pathology in the Brigham and Women's Hospital, Boston, Mass. Ethnic groups and hair color were not discriminated in the selection of samples.

Immunohistochemical studies were performed using formalin fixed paraffin-embedded tissue. Sections were cut at 4 micrometers, dried at 37° C., deparaffinized in xylenes, and hydrated in a graded series of alcohols. Undiluted monoclonal antibody D5 specific for MITF (J. Du et al., Am J Pathol 163:333-43 (2003)) was used to stain for MITF. Hybridoma culture supernatant was used because other sources proved less reliable. Microwave antigen retrieval for D5 immunostaining was performed using 10 mM citrate buffer at pH 6.0 for 30 minutes at 93° C. followed by a 10 minute cool down period. All staining was performed using DAKO Envision+ detection system (Alkaline Phosphatase) and Fuchsin as the chromogen. Only a nuclear pattern of D5 staining was regarded as positive.

TUNEL Assays. In situ cell death was detected by TUNEL staining (TdT-mediated dUTP-digoxigenin nick end labeling technique) using the “in situ cell death detection kit” (Roche) combined with Alex488-conjugated anti-fluorescein antibodies (Molecular Probes, Inc., Eugene, Oreg.).

### Example 2

### Hair Graying in Bcl2^{-/-} Mice

This example demonstrates that differentiated melanocytes are lost in the hair bulb of Bcl-2 deficient mice. Bcl-2^{-/+} (C57BL/6J) and Tyr^{c-2j/c-2j} (C57BL6J) mice were purchased from the Jackson Laboratory, Bar Harbor, Me. Dct-lacZ transgenic mice were obtained as gift from I. Jackson. The Dct-LacZ transgenic colony (CBA/C57BL6) was backcrossed to C57BL/6J.

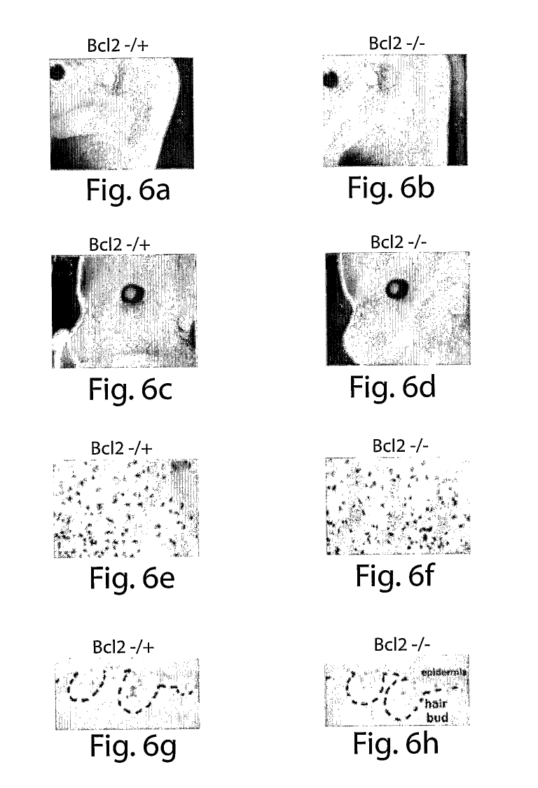

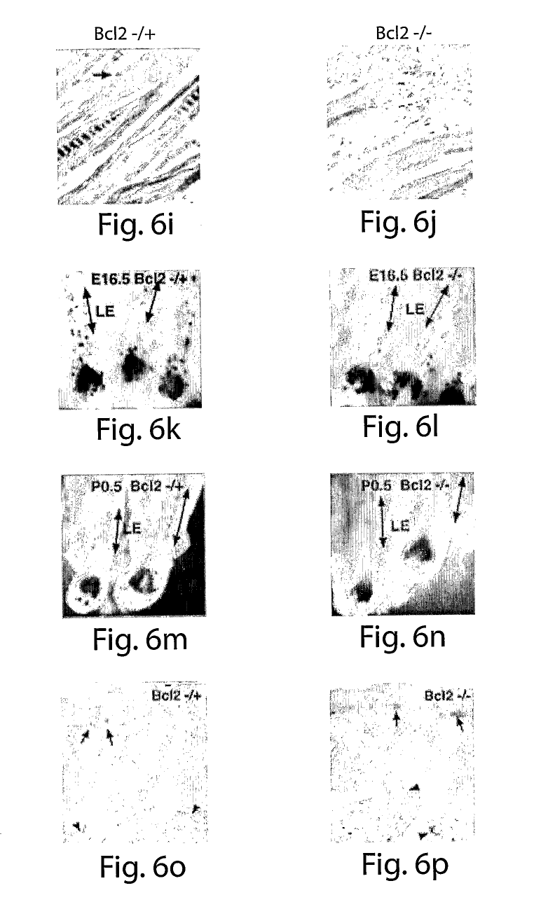

As mentioned above, hair-graying in the Bcl2^{-/-} background has been suggested to arise by chemical cytotoxity of melanin synthesis. Distribution and morphology of melanoblasts among Bcl2^{-/-}, Bcl2^{-/+} and Bcl2^{+/+} mice were normal during early development (FIGS. 6a-6h). Bcl2^{-/-} mice gray after the first hair molting (FIGS. 1b and 1c) with white hairs. Histologically, differentiated melanocytes were almost completely absent in Bcl2^{-/-} pelage (body hair) or whisker follicles (FIGS. 1e, 1g) as compared to Bcl2^{-/+} (FIGS. 1d, 1f, 1h) or Bcl2^{+/+} follicles at postnatal day 39 (P39). As shown in FIG. 1, albino background (Tyr^{c-2j/c-2j}, C57BL/6J) did not protect against melanocyte loss in Bcl2^{-/-} mice, suggesting that melanin synthesis is unnecessary for this melanocyte disappearance. Of note, Bcl2^{-/-} follicles in the second hair cycle lacked both differentiated melanocytes in the hair bulb (“Bb”) and undifferentiated Dct-lacZ⁺ melanoblasts in the stem-cell niche (located at the bulge area (“Bg”) in pelage follicles) (FIGS. 1d, 1e, 6i and 6j), suggesting that Bcl2 might be important for survival of melanocyte stem cells.

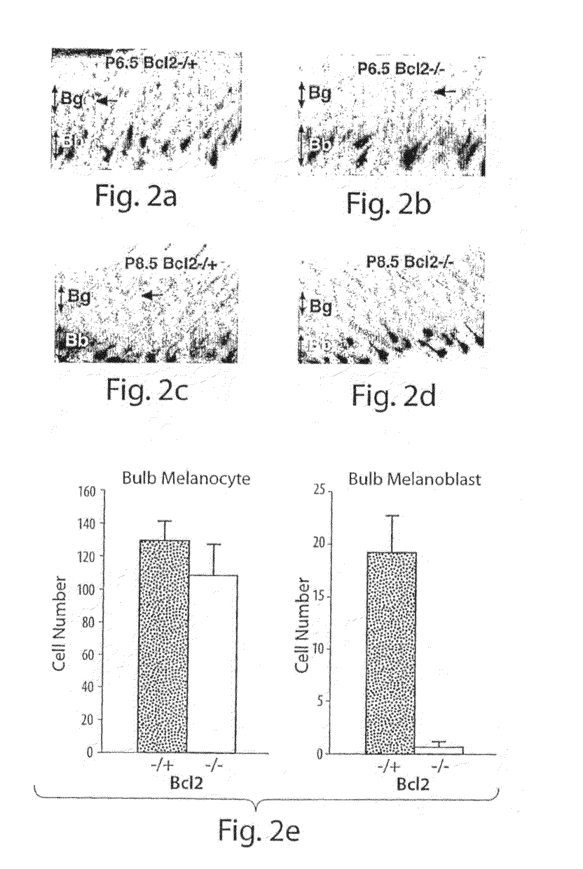

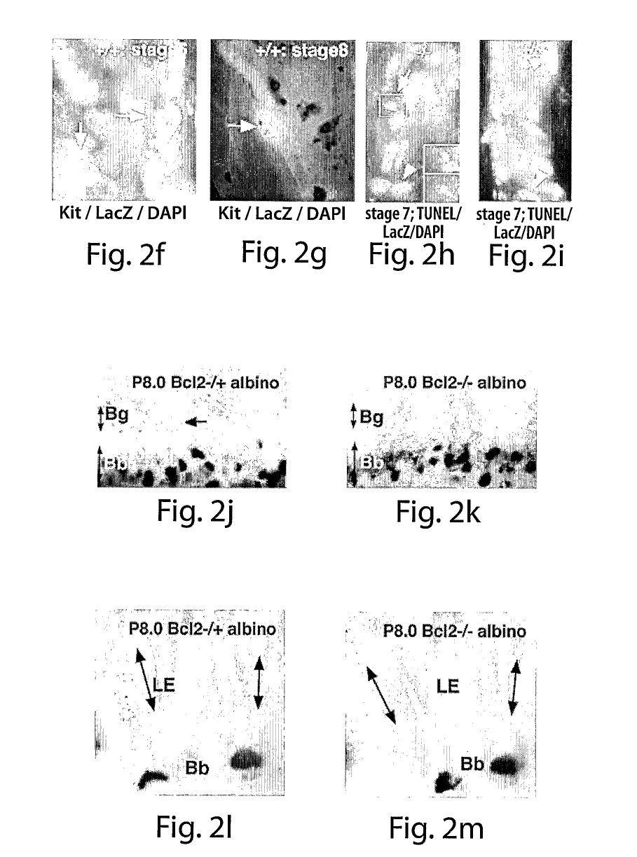

Looking earlier, at postnatal day P6.5 when hair follicle morphogenesis is almost complete, Bcl2^{-/-} Bcl2^{-/+} follicles appeared normal (FIG. 2b). In contrast, Bcl2^{-/-} follicles at P8.5 showed sudden, virtually complete loss of melanoblasts in the niche (bulge area, FIG. 2d), while the number of melanocytes in the hair bulb did not show significant differences between Bcl2^{-/+} and Bcl2^{-/-} mice (FIG. 2e). In both pelage and whisker follicles from Bcl2^{-/-} animals, disappearance of niche melanoblasts began at stage 6 of hair follicle morphogenesis (FIGS. 6k-6n), and by stage 8, they were gone (standardized hair follicle stages based on R. Paus et al., J Invest Dermatol 113:523-32 (1999)). At this stage niche melanoblasts undergo a morphologic change from a dendritic shape into a slender, oval shape with shrinkage to maximal nuclear/cytoplasmic ratio upon entry into the dormant state (FIGS. 2f, 2g). This change in morphology was seen cyclically at corresponding stages of subsequent cycles.

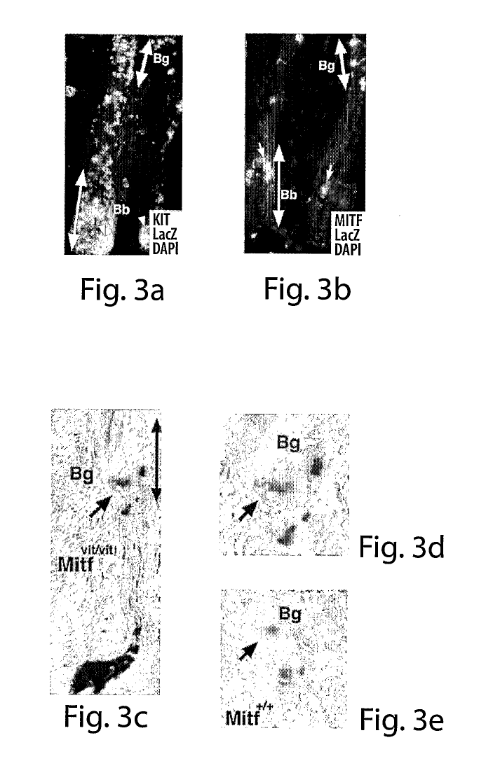

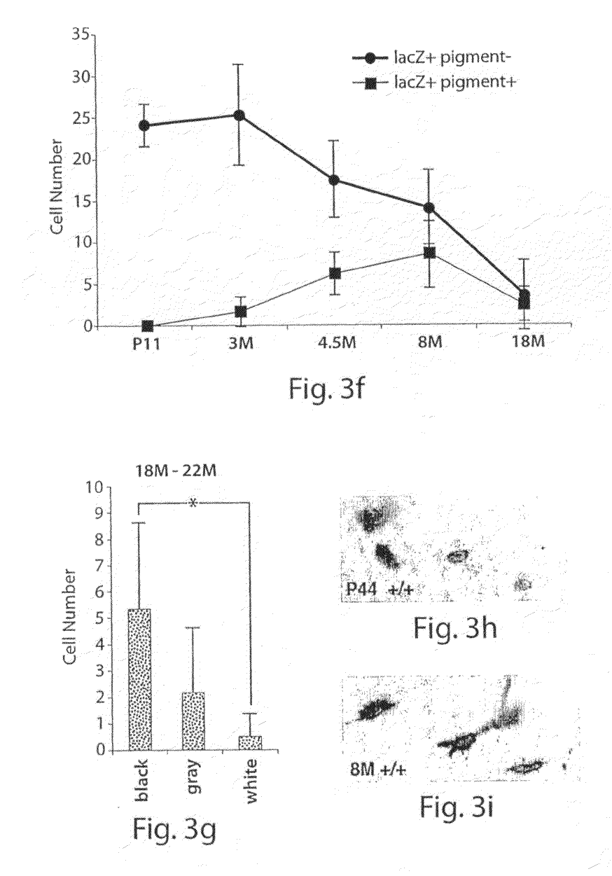

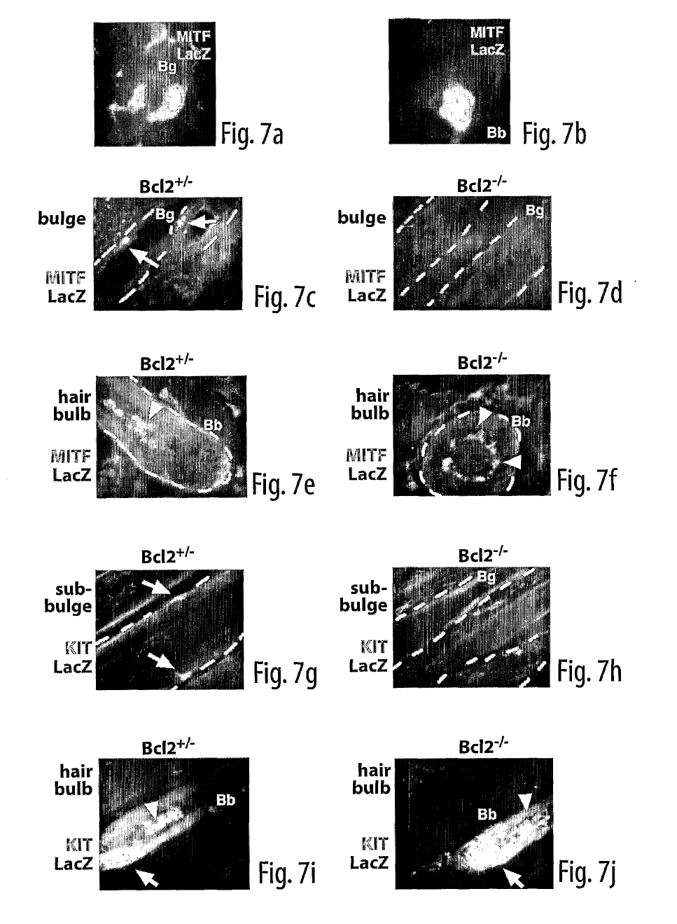

Apoptosis of melanocyte stem cells was observed at the same stage on the albino background (Tyr^{c-2j/c-2j}) both in pelage and whisker hair follicles (FIGS. 2h-2m). The same pattern of cell loss was detected using Dct-LacZ, KIT, or MITF as markers (FIGS. 3a, 3b, 7). On the other hand, melanocytes in the epidermis and dermis of hairless skin (e.g. tail and soles) survived throughout the hair regeneration cycle (FIGS. 6o and 6p). These findings indicate that BCL2 selectively protects melanocyte stem cells at the time of their transition into the dormant state in the niche and could potentially be responsible for certain forms of human pre-senile hair graying.

### Example 3

### Hair Graying in Mitf-vit Mutant Mice

This example demonstrates the effect of a Mitf mutation on melanocyte stem cells. Mitf^{vit/vit} (C57BL/6) mice were obtained as a gift from M. L. Lamoreux.

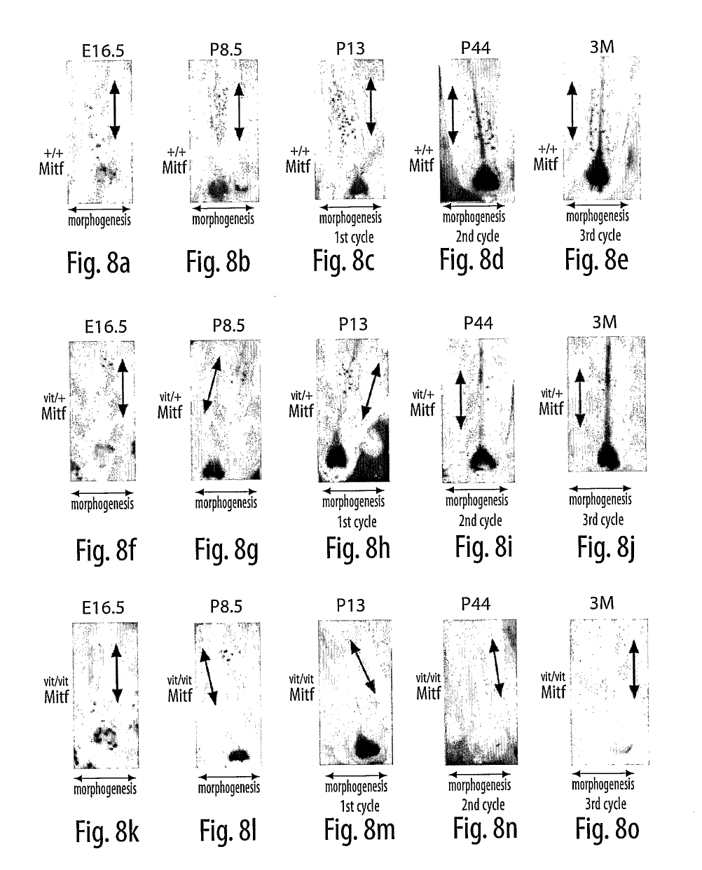

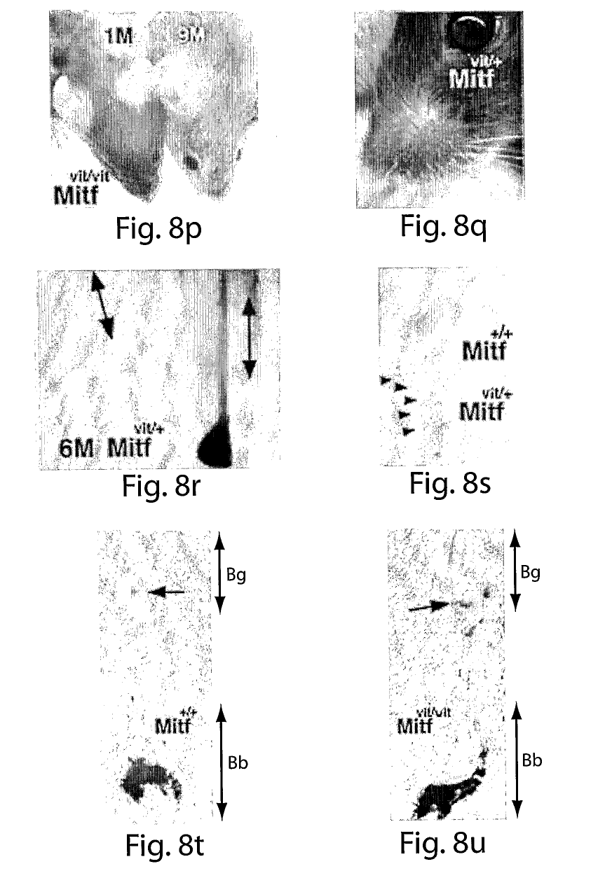

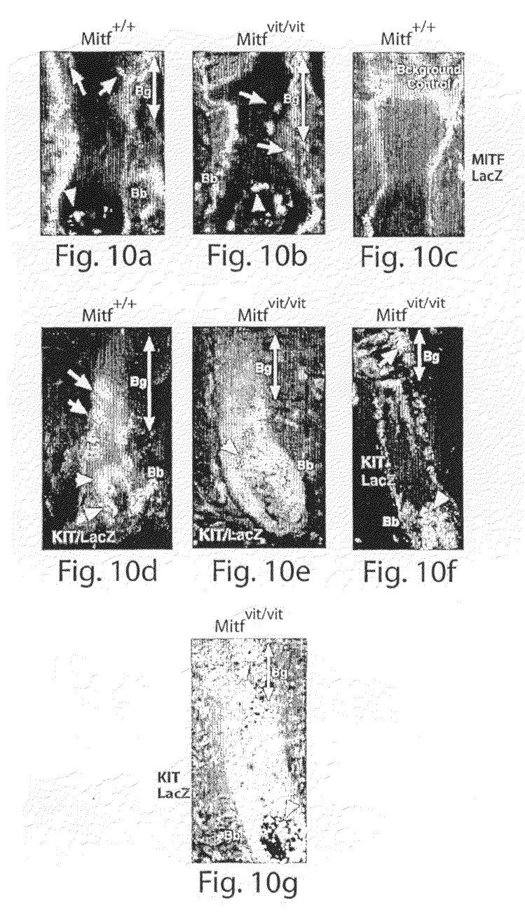

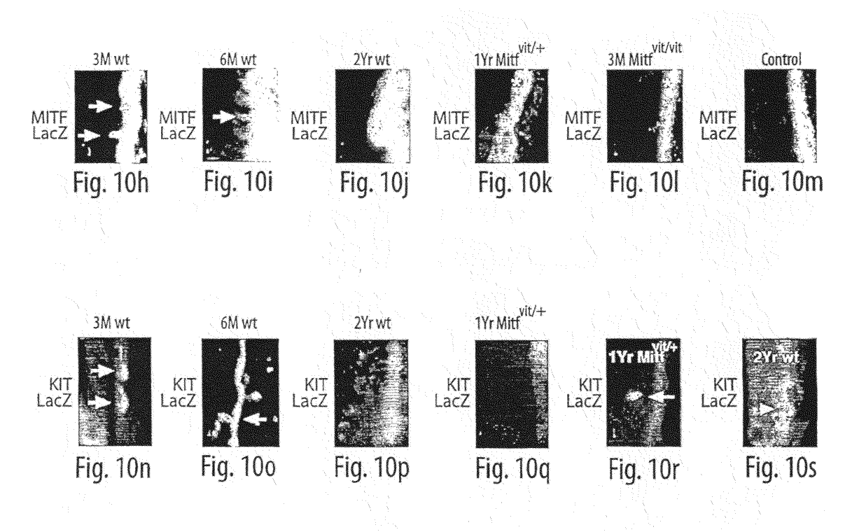

In contrast to Bcl2^{-/-} (Example 2), the Mitf^{vit/vit} (A. B. Lerner et al., J Invest Dermatol 87:299-304 (1986)) graying mouse model exhibited a gradual decrease of melanocyte stem cells, rather than abrupt loss (FIGS. 8, 10). This strain contains a mild hypomorphic mutation in Mitf, the melanocyte master transcriptional regulator. E. Steingrimsson, N. G. Copeland, N. A. Jenkins, Annu Rev Genet (2004); E. R. Price, D. E. Fisher, Neuron 30:15-8 (2001), and references therein). At early-mid-anagen of the third hair cycle lacZ⁺ cells left in the niche of Mitf^{vit/vit} pelage follicles and Mitf^{vit/+} whisker follicles often produced melanin pigment and exhibited a bipolar or dendritic morphology (FIGS. 3c, 3d, 8j, 8s). These pigmented cells are unusual because the niche of wildtype controls contains only unpigmented melanocyte stem cells. The term “ectopic pigmentation or differentiation” is used for this reproducibly observed population because it is uncertain by which pathway these cells became pigmented, although they were absent in age-matched controls whose niche melanoblasts remain undifferentiated (FIGS. 3e, 8u).

### Example 4

### Hair Graying in Aging Wildtype Mice

This example demonstrates the effect of aging on melanocyte stem cells in mice. Wildtype C57BL/6J mice were obtained from the Jackson Laboratory, Bar Harbor, Me.

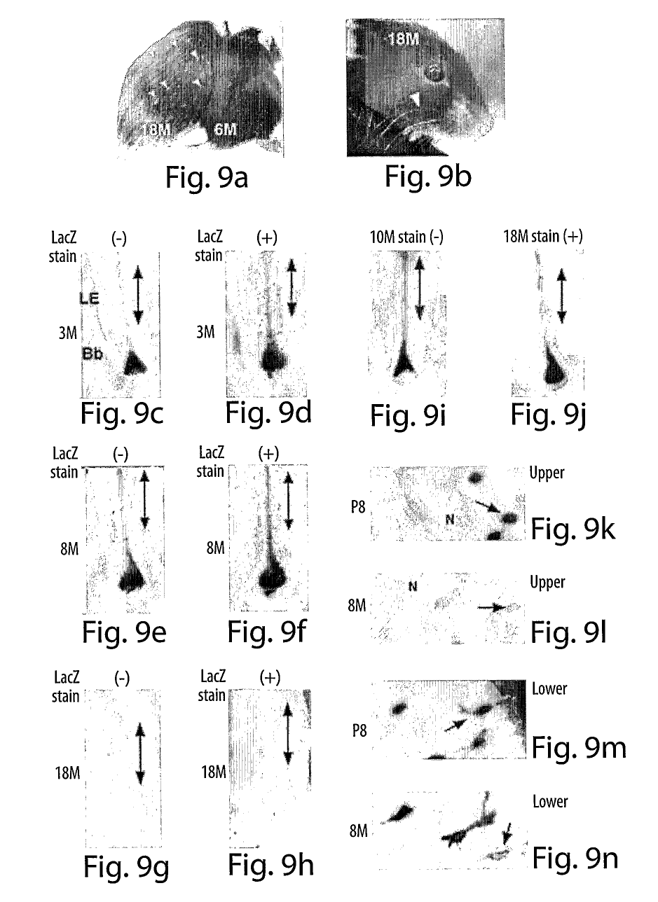

Physiologic (senile) aging in mice also produces hair graying (FIG. 9), which was found to be due to loss of melanocyte stem cells. Indeed during physiologic aging, niche melanoblasts (LacZ⁺) were lost in a gradual and progressive fashion (FIGS. 3f, 3g). Moreover whole-mount cross-sections of 8-month-old follicles revealed pigment-containing melanocytes within the stem-cell niche in addition to their scattered distribution in the outer root sheath below the niche in whisker follicles (FIGS. 3h, 3i, 9k-9n). The appearance of these pigmented melanocytes in the niche is reminiscent of pigmented niche melanocytes observed during the accelerated graying of Mitf-vit mutants (Example 3). Quantitative analysis revealed that the presence of these cells was accompanied by simultaneous loss of the typical unpigmented Dct-lacZ⁺ melanoblasts in the niche and correlated closely with aging (FIGS. 3f, 3g). Thus self-maintenance of melanocyte stem cells is essentially complete in young animals but becomes defective with aging.

### Example 5

### Hair Graying in Aging Humans

This example demonstrates the effect of aging on distribution of melanocyte stem cells and melanocytes in human scalp hair follicles.

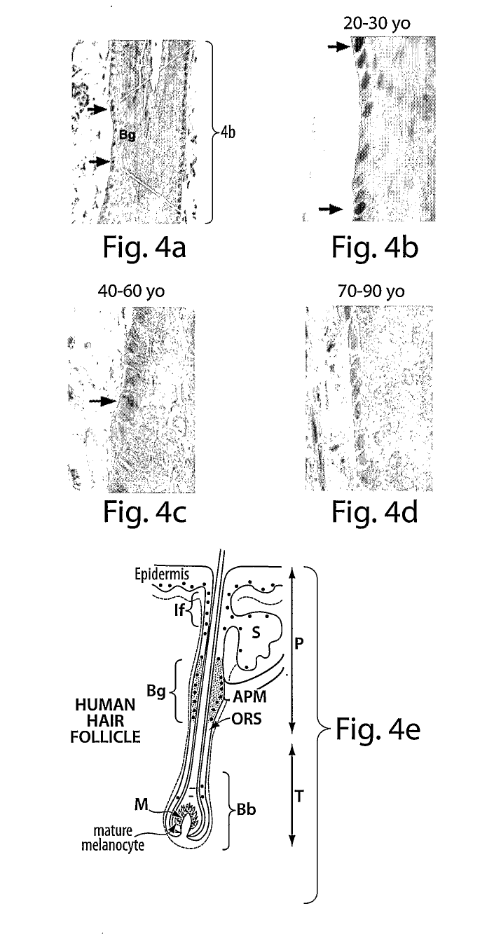

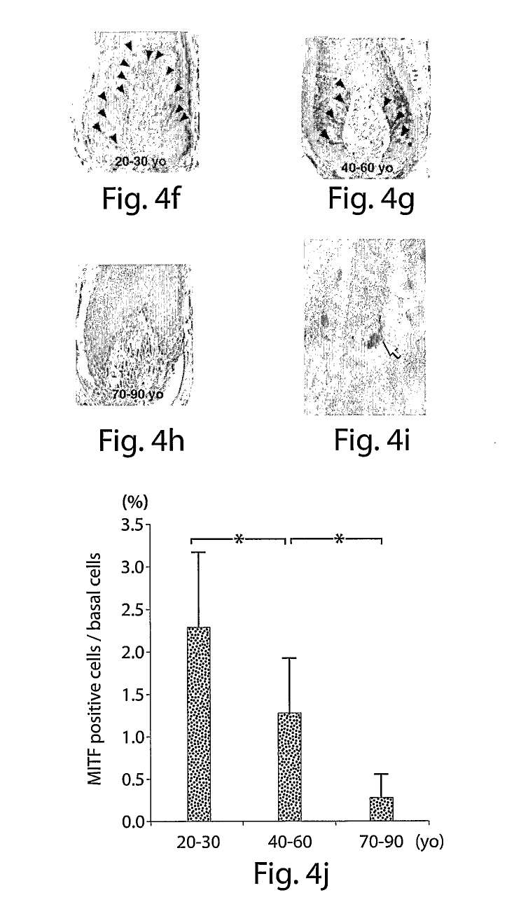

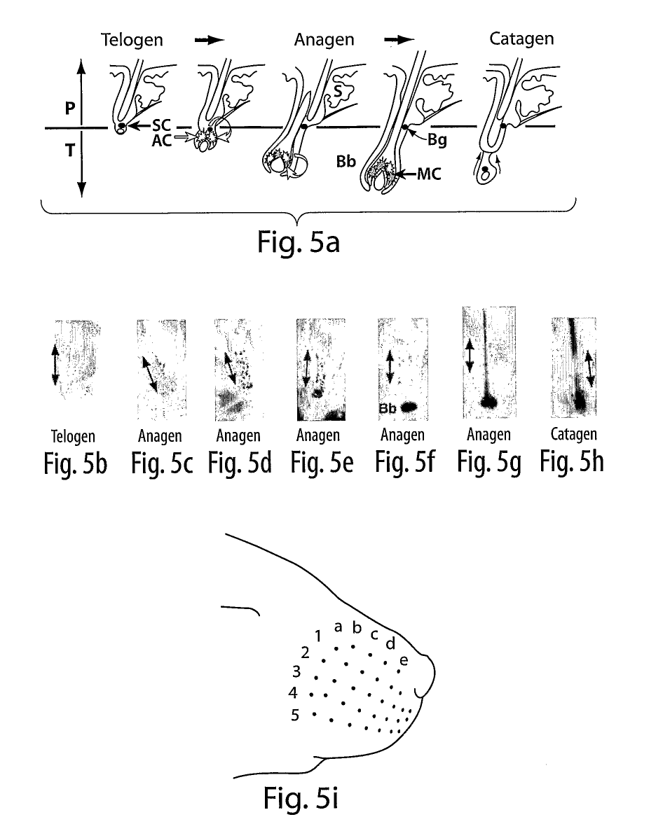

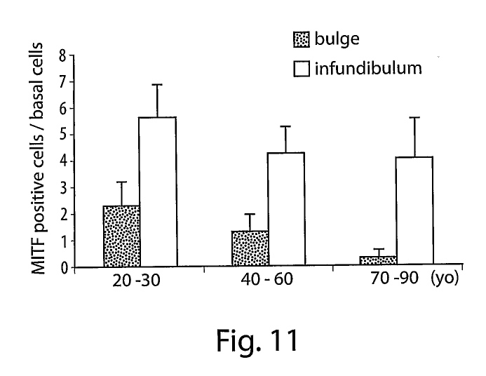

The distribution of melanoblasts was analyzed in aging human hair follicles using MITF immunostaining (FIG. 4). MITF⁺ small unpigmented melanoblasts were found in the outer root sheath preferentially around the bulge area where the arrector pili muscle attaches below the level of the sebaceous gland (FIGS. 4a-4c), similar to previously described “amelanotic melanocytes” (W. Montagna, H. B. Chase, Am J Anat 99:415-446 (1956); R. G. Staricco, Ann N Y Acad Sci 100:239-55 (1963)) which express PMEL17 (T. Horikawa et al., J Invest Dermatol 106:28-35 (1996); S. Commo, B. A. Bernard, Pigment Cell Res 13:253-9 (2000)), a transcriptional target of MITF (J. Du et al., Am J Pathol 163:333-43 (2003)). These cells are suggested to be a reservoir population for differentiated melanocytes (R. G. Staricco, Ann N Y Acad Sci 100:239-55 (1963)) and exhibited very similar morphology to the corresponding cells in mice. While MITF⁺ immature melanoblasts were abundant in follicles from 20-30 year olds (2-3% of the total basal keratinocytes in the bulge area), they were absent from most hair follicles of 70-90 year olds (FIG. 4j). MITF⁺ melanocytes in the uppermost area (infundibulum) of the outer root sheath did not decrease significantly with aging, thus serving as a control population in these studies (FIG. 11).

Follicles from intermediate aged individuals (40-60 years old) revealed intermediate loss of bulge melanoblasts (FIGS. 4c, 4j). Bulge melanoblasts were found more in pigmented follicles than in gray follicles, as shown recently with PMEL17⁺ bulge melanoblasts of middle-aged individuals. S. Commo, O. Gaillard, B. A. Bernard, Br J Dermatol 150:435-43 (2004). In addition, as with aged or Mitf^{vit} mouse follicles, ectopically pigmented MITF⁺ cells were occasionally observed in the bulge area or just below. These cells closely resembled the “dendritic melanocytes” described by Narisawa et al. in the bulge area of human follicles. Y. Narisawa, H. Kohda, T. Tanaka, Acta Derm Venereol 77, 97-101 (1997). The ectopically pigmented or differentiated melanocytes were seen exclusively in middle-aged follicles, but did not accumulate in the bulge area, suggesting that they are not self-maintaining.

### Example 6

### In Vivo Screening for Inhibitors of Melanocyte Stem Cell Loss

Bcl-2^{-/-}, Bcl-2^{-/+}, Mitf^{vit/vit} , Mitf^{vit/+}, Bcl-2^{-/+}X Mitf^{vit/vit}, Bcl-2^{-/+} X Mitf^{vit/+}, or wildtype control mice are administered test agents. Mice are observed for development of hair/whisker graying. Histologic analysis of follicles is performed as described in previous examples. Inhibition of melanocyte stem cell loss compared to appropriate control is interpreted to identify a test agent as an inhibitor of melanocyte stem cell loss.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for inhibiting melanocyte stem cell loss, the method comprising
contacting melanocyte stem cells with an agent that inhibits melanocyte stem cell loss.

2. The method of claim 1, wherein the agent that inhibits melanocyte stem cell loss comprises a cytokine.

3. The method of claim 1, wherein the agent that inhibits melanocyte stem cell loss comprises an agent that upregulates MITF.

4. The method of claim 1, wherein the agent that inhibits melanocyte stem cell loss comprises MITF.

5. The method of claim 1, wherein the agent that inhibits melanocyte stem cell loss comprises a nucleic acid encoding MITF.

6. The method of claim 1, wherein the agent that inhibits melanocyte stem cell loss comprises an agent that upregulates Bcl-2.

7. The method of claim 1, wherein the agent that inhibits melanocyte stem cell loss comprises a Bim antagonist.

8. The method of claim 1, wherein the agent that inhibits melanocyte stem cell loss is selectively targeted for delivery to melanocyte stem cells.

9. The method of claim 8, wherein the agent that inhibits melanocyte stem cell loss is conjugated to a Kit ligand.

10. A method for treating a subject having loss of natural hair pigment, the method comprising
administering to the subject an agent that inhibits melanocyte stem cell loss in the subject.

11. The method of claim 10, wherein the agent that inhibits melanocyte stem cell loss comprises a cytokine.

12. The method of claim 10, wherein the agent that inhibits melanocyte stem cell loss comprises an agent that upregulates MITF.

13. The method of claim 10, wherein the agent that inhibits melanocyte stem cell loss comprises MITF.

14. The method of claim 10, wherein the agent that inhibits melanocyte stem cell loss comprises a nucleic acid encoding MITF.

15. The method of claim 10, wherein the agent that inhibits melanocyte stem cell loss comprises an agent that upregulates Bcl-2.

16. The method of claim 10, wherein the agent that inhibits melanocyte stem cell loss comprises a Bim antagonist.

17. The method of claim 10, wherein the agent that inhibits melanocyte stem cell loss is selectively targeted for delivery to melanocyte stem cells.

18. The method of claim 17, wherein the agent that inhibits melanocyte stem cell loss is conjugated to a Kit ligand.

19. The method of claim 10, wherein the administering comprises topically administering.

20. The method of claim 10, wherein the administering comprises systemically administering.

21. A method for identifying an agent that inhibits melanocyte stem cell loss, the method comprising
placing into culture a defined number of isolated epidermal melanocytes or isolated melanocyte stem cells, under conditions that simulate stem cell environment, for a defined duration in presence of a test agent;
measuring a test response, wherein the test response is a number of epidermal melanocytes or melanocyte stem cells cultured for the defined duration in the presence of the test agent;
comparing the test response to a control response, wherein the control response is a number of isolated epidermal melanocytes or isolated melanocyte stem cells, cultured beginning with the defined number of said cells, under the conditions that simulate stem cell environment for the defined duration in absence of the test agent; and
identifying the test agent as an agent that inhibits melanocyte stem cell loss when the test response exceeds the control response.

22. The method of claim 21, wherein the isolated epidermal melanocytes or isolated melanocyte stem cells are isolated epidermal melanocytes.

23. The method of claim 21, wherein the isolated epidermal melanocytes or isolated melanocyte stem cells are isolated melanocyte stem cells.

24. The method of claim 21, wherein the isolated epidermal melanocytes or isolated melanocyte stem cells are deficient for Bcl-2.

25. The method of claim 21, wherein the isolated epidermal melanocytes or isolated melanocyte stem cells are deficient for MITF.

26. The method of claim 21, wherein the test agent comprises a cytokine.

27. The method of claim 21, wherein the test agent comprises an agent that upregulates MITF.

28. The method of claim 21, wherein the test agent comprises a nucleic acid encoding MITF.

29. The method of claim 21, wherein the test agent comprises an agent that upregulates Bcl-2.

30. The method of claim 21, wherein the test agent comprises a Bim antagonist.

31. The method of claim 21, wherein the test agent is selectively targeted for delivery to epidermal melanocytes or melanocyte stem cells.

32. The method of claim 31, wherein the test agent is conjugated to a Kit ligand.

33. A method for identifying an agent that inhibits ectopic pigmentation or differentiation of melanocyte stem cells in a subject, the method comprising
administering a test agent to the subject;
measuring a test response to the test agent, wherein the measuring comprises assessing ectopic pigmentation or differentiation of melanocyte stem cells in the subject;
comparing the test response to a control response; and
identifying the test agent as an agent that inhibits ectopic pigmentation or differentiation of melanocyte stem cells in the subject when the control response exceeds the test response.

34. The method of claim 33, wherein the test agent comprises a cytokine.

35. The method of claim 33, wherein the test agent comprises an agent that upregulates MITF.

36. The method of claim 33, wherein the test agent comprises a nucleic acid encoding MITF.

37. The method of claim 33, wherein the test agent comprises an agent that upregulates Bcl-2.

38. The method of claim 33, wherein the test agent comprises a Bim antagonist.

39. The method of claim 33, wherein the test agent is selectively targeted for delivery to melanocyte stem cells.

40. The method of claim 39, wherein the test agent is conjugated to a Kit ligand.

41. The method of claim 33, wherein the administering comprises topically administering.

42. The method of claim 33, wherein the administering comprises systemically administering.

43. A method for identifying an agent useful for treating a subject having loss of natural hair pigment, the method comprising
placing into culture a defined number of isolated epidermal melanocytes or isolated melanocyte stem cells, under conditions that simulate stem cell environment, for a defined duration in presence of a test agent;
measuring a test response, wherein the test response is a number of epidermal melanocytes or melanocyte stem cells cultured for the defined duration in the presence of the test agent;
comparing the test response to a control response, wherein the control response is a number of isolated epidermal melanocytes or isolated melanocyte stem cells, cultured beginning with the defined number of said cells, under the conditions that simulate stem cell environment for the defined duration in absence of the test agent; and
identifying the test agent as an agent useful for treating a subject having loss of natural hair pigment when the test response exceeds the control response.

44. The method of claim 43, wherein the isolated epidermal melanocytes or isolated melanocyte stem cells are isolated epidermal melanocytes.

45. The method of claim 43, wherein the isolated epidermal melanocytes or isolated melanocyte stem cells are isolated melanocyte stem cells.

46. The method of claim 43, wherein the isolated epidermal melanocytes or isolated melanocyte stem cells are deficient for Bcl-2.

47. The method of claim 43, wherein the isolated epidermal melanocytes or isolated melanocyte stem cells are deficient for MITF.

48. The method of claim 43, wherein the test agent comprises a cytokine.

49. The method of claim 43, wherein the test agent comprises an agent that upregulates MITF.

50. The method of claim 43, wherein the test agent comprises a nucleic acid encoding MITF.

51. The method of claim 43, wherein the test agent comprises an agent that upregulates Bcl-2.

52. The method of claim 43, wherein the test agent comprises a Bim antagonist.

53. The method of claim 43, wherein the test agent is selectively targeted for delivery to epidermal melanocytes or melanocyte stem cells.

54. The method of claim 53, wherein the test agent is conjugated to a Kit ligand.

55. A method for identifying an agent that inhibits melanocyte stem cell loss in a subject, the method comprising
administering a test agent to the subject;
measuring a test response to the test agent, wherein the measuring comprises counting melanocyte stem cells or assessing capacity of the melanocyte stem cells to produce melanocytes after entry into growth phase;
comparing the test response to a control response; and
identifying the test agent as an agent that inhibits melanocyte stem cell loss in the subject when the test response exceeds the control response.

56. The method of claim 55, wherein the test agent comprises a cytokine.

57. The method of claim 55, wherein the test agent comprises an agent that upregulates MITF.

58. The method of claim 55, wherein the test agent comprises a nucleic acid encoding MITF.

59. The method of claim 55, wherein the test agent comprises an agent that upregulates Bcl-2.

60. The method of claim 55, wherein the test agent comprises a Bim antagonist.

61. The method of claim 55, wherein the test agent is selectively targeted for delivery to melanocyte stem cells.

62. The method of claim 61, wherein the test agent is conjugated to a Kit ligand.

63. The method of claim 55, wherein the administering comprises topically administering.

64. The method of claim 55, wherein the administering comprises systemically administering.
